(19) United States
(12) Patent Application Publication
(10) Pub. No.: US 2025/0114031 A1
(43) Pub. Date: Apr. 10, 2025

(54) USER-TRAINED SYSTEM

(71) Applicant: TRINAMIX GMBH, Ludwigshafen Am Rhein (DE)

(72) Inventors: Christian HESS, Ludwigshafen am Rhein (DE); Vera Charlotte KOCKLER, Ludwigshafen am Rhein (DE); Christian LENNARTZ, Ludwigshafen am Rhein (DE); Friedrich SCHICK, Muenchen (DE); Jonas GROSSE HOLTHAUS, Bergisch-Gladbach (DE)

(21) Appl. No.: 18/727,747

(22) PCT Filed: Feb. 13, 2023
(86) PCT No.: PCT/EP23/53421

§ 371 Date: Jul. 10, 2024

(30) Foreign Application Priority Data
Feb. 15, 2022 (EP) 22156778.7

(51) Int. Cl. A61B 5/00; G06T 7/00

(52) U.S. Cl. CPC A61B 5/441; A61B 5/0077; G06T 7/0012; G06T 2207/20081; G06T 2207/20084; G06T 2207/20132; G06T 2207/30088

(57) ABSTRACT

Disclosed herein is a method for training a data-driven model for determining a user-selected parameter value related to the condition of a human including:
(a) receiving an image of a body part of the human while the body part is illuminated by a light pattern containing at least one pattern feature,
(b) determining skin pattern features from the image, where a skin pattern feature is a pattern feature which has been reflected by skin,
(c) receiving from a user interface a user-selected parameter value related to the condition of the human, and
(d) training a data-driven model with a training dataset comprising the skin pattern features and the user-selected parameter value.

The present invention is in the field of image processing for the monitoring the condition of a human. In particular, it relates to a method for training a data-driven model for determining a user-selected parameter related to the condition of a human, a method for determining a user-selected parameter related to the condition of a human, non-transitory computer-readable data medium storing a computer program including instructions for executing steps of the method and a device for determining a user-selected parameter related to the condition of a human.

### BACKGROUND

Modern portable devices provide many ways of monitoring human activity or health. For example, fitness trackers record body functions such as heart rate or blood pressure. More and more analytic applications are added to derive a fitness level or a health condition.

US 2021/0 397 929 discloses a wearable device having sensors for measurements associated with a person. The device uses deep learning for intelligent monitoring.

However, for any given use case, the provider of the portable device has to adjust hardware and software to enable such use case. Often, data-driven models are applied which have been trained by historic datasets according to a specific use case. The provider of the device has to collect such historic datasets and train the model. Hence, for every user, the model works identically, so no personal deviations from an average person contributing to the historic dataset are taken into account.

It was hence the object of the present invention to overcome these shortcomings. In particular, a device was aimed at which provides more flexibility for the user to analyze the conditions of his body. Such analysis should be made available taken into account his individual situation. At the same time the device should be easy to handle and require little effort for setting up a use case until it can produce meaningful results.

### SUMMARY

These objects were achieved by the present invention. In one aspect it relates to a method for training a data-driven model for determining a user-selected parameter value related to the condition of a human comprising:
(a) receiving an image of a body part of the human while the body part is illuminated by a light pattern containing at least one pattern feature,
(b) determining skin pattern features from the image, wherein a skin pattern feature is a pattern feature which has been reflected by skin,
(c) receiving from a user interface a user-selected parameter value related to the condition of the human,
(d) training a data-driven model with a training dataset comprising the skin pattern features and the user-selected parameter value.

In another aspect, the invention relates to a method for determining a user-selected parameter value related to the condition of a human comprising:
(a) receiving an image of a body part of the human while the body part is illuminated by a light pattern containing at least one pattern feature,
(b) determining skin pattern features from the image, wherein a skin pattern feature is a pattern feature which has been reflected by skin,
(c) determining the user-selected parameter value related to the condition of a human from the skin pattern features by using a data-driven model which has been trained by a set of historic data comprising skin pattern features and the user-selected parameter value, and
(d) outputting the user-selected parameter value.

In another aspect, the invention relates to a non-transitory computer-readable data medium storing a computer program including instructions for executing steps of the method according to any one of the preceding claims.

In another aspect, it relates to a use of data-driven model for determining a user-selected parameter value related to the condition of a human.

In another aspect, the invention relates to a device for determining a user-selected parameter value related to the condition of a human comprising:
(a) a projector for projecting patterned light containing at least one pattern feature onto a body part of the human,
(b) a camera for recording an image of the body part while it is illuminated by patterned light,
(c) a user interface for receiving a user-selected parameter value related to the condition of the human,
(d) a processor for
   determining skin pattern features from the image, wherein a skin pattern feature is a pattern feature which has been reflected by skin
   training a data-driven model with a training dataset comprising the skin pattern features and the user-selected parameter value.

The present invention provides means for a user to set up his own monitoring of the condition of his body. By illuminating the body part with patterned light and only taking into account those pattern features which have been reflected by skin, the amount of training data is reduced and higher reliability is obtained. Disturbances like wearing glasses or a hat hence do not require dedicated training data sets, but are ignored by the method of the present invention. In addition, the input for a data-driven model is standardized for each use case, hence allowing more flexibility for the user. He is hence not limited to methods the provider of a device has set up, but can set up use cases himself. Also, the data-driven model can be exactly trained for a particular user, hence any user-characteristics, for example skin color, age or diseases like allergies, can be taken into account.

### DETAILED DESCRIPTION

In one aspect, the present invention relates to a method for training a data-driven model. The method comprises receiving an image of a body part of the human. The image of the body part should show the body part at least partially uncovered, i.e. at least part of the body part shows exposed skin. To obtain good results, the body part should be essentially uncovered or fully uncovered. Any body part is conceivable, for example a face, a hand, an arm, a leg, a foot. Also, more detailed parts are possible, for example an eye, an ear, a nose, or a finger. The image can show one body part or more than one, for example two or three. For example, if the body part is a hand, the image may show both hands of the person. For each use case, which means for each purpose the data-driven model is intended to be used, it is preferable that each image shows the same body part. However, it is also possible that images show different body parts if the information of interest is contained in any of the chosen body parts. It is possible that one image of a body part is received at a time or more than one. More than one images may be received, for example, from slightly different angles like images obtained from a stereo camera or images recorded within a short time interval, for example 1 second or less, showing subtle time changes like motion. Two or more such images may be further processed separately or merged into a composite image for further processing.

The body part is illuminated by a light pattern containing at least one pattern feature. The illumination can be achieved by using a projector or illumination source which emits the light pattern onto the body part. The illumination source may comprise at least one light source. The illumination source may comprise a plurality of light sources. The illumination source may comprise an artificial illumination source, in particular at least one laser source and/or at least one incandescent lamp and/or at least one semiconductor light source, for example, at least one light-emitting diode, in particular an organic and/or inorganic light-emitting diode. As an example, the light emitted by the illumination source may have a wavelength of 300 to 1100 nm, especially 500 to 1100 nm. Additionally or alternatively, light in the infrared spectral range may be used, such as in the range of 780 nm to 3.0 μm. Specifically, the light in the part of the near infrared region where silicon photodiodes are applicable specifically in the range of 700 nm to 1100 nm may be used. Using light in the near infrared region allows that light is not or only weakly detected by human eyes and is still detectable by silicon sensors, in particular standard silicon sensors. The illumination source may be adapted to emit light at a single wavelength. In other embodiments, the illumination may be adapted to emit light with a plurality of wavelengths allowing additional measurements in other wavelengths channels. The light source may be or may comprise at least one multiple beam light source. For example, the light source may comprise at least one laser source and one or more diffractive optical elements (DOEs).

Specifically, the illumination source may comprise at least one laser and/or laser source. Various types of lasers may be employed, such as semiconductor lasers, double heterostructure lasers, external cavity lasers, separate confinement heterostructure lasers, quantum cascade lasers, distributed bragg reflector lasers, polariton lasers, hybrid silicon lasers, extended cavity diode lasers, quantum dot lasers, volume Bragg grating lasers, Indium Arsenide lasers, transistor lasers, diode pumped lasers, distributed feedback lasers, quantum well lasers, interband cascade lasers, Gallium Arsenide lasers, semiconductor ring laser, extended cavity diode lasers, or vertical cavity surface-emitting lasers. Additionally or alternatively, non-laser light sources may be used, such as LEDs and/or light bulbs. The illumination source may comprise one or more diffractive optical elements (DOEs) adapted to generate the illumination pattern. For example, the illumination source may be adapted to generate and/or to project a cloud of points, for example the illumination source may comprise one or more of at least one digital light processing projector, at least one LCoS projector, at least one spatial light modulator; at least one diffractive optical element; at least one array of light emitting diodes; at least one array of laser light sources. On account of their generally defined beam profiles and other properties of handleability, the use of at least one laser source as the illumination source is particularly preferred. The illumination source may be integrated into a housing of the device for executing the training method.

The illumination source may be configured for generating at least one illumination pattern for illumination of the body part. The illumination pattern may comprise at least one pattern selected from the group consisting of: at least one point pattern, in particular a pseudo-random point pattern; a random point pattern or a quasi-random pattern; at least one Sobol pattern; at least one quasiperiodic pattern; at least one pattern comprising at least one pre-known feature; at least one regular pattern; at least one triangular pattern; at least one hexagonal pattern; at least one triclinic pattern; at least one rectangular pattern at least one pattern comprising convex uniform tilings; at least one line pattern comprising at least one line; at least one line pattern comprising at least two lines such as parallel or crossing lines.

As used herein, the term “pattern” refers to an arbitrary known or pre-determined arrangement comprising at least one arbitrarily shaped feature. The pattern may comprise at least one feature such as a point or symbol. The pattern may comprise a plurality of features. The pattern may comprise an arrangement of periodic or non-periodic features. As used herein, the term “at least one illumination pattern” refers to at least one arbitrary pattern comprising at least one illumination feature adapted to illuminate at least one part of the object. As used herein, the term “illumination feature” refers to at least one at least partially extended feature of the pattern. The illumination pattern may comprise a single illumination feature. The illumination pattern may comprise a plurality of illumination features. For example, the illumination pattern may comprise at least one line pattern. For example, the illumination pattern may comprise at least one stripe pattern. For example, the illumination pattern may comprise at least one checkerboard pattern. For example, the illumination pattern may comprise at least one pattern comprising an arrangement of periodic or non-periodic features. The illumination pattern may comprise regular and/or constant and/or periodic pattern such as a triangular pattern, a rectangular pattern, a hexagonal pattern, a triclinic pattern or a pattern comprising further convex tilings. The illumination pattern may exhibit the at least one illumination feature selected from the group consisting of: at least one point; at least one line; at least two lines such as parallel or crossing lines; at least one point and one line; at least one arrangement of periodic or non-periodic feature; at least one arbitrary shaped featured. For example, the illumination source may be adapted to generate and/or to project a cloud of points. A distance between two features of the illumination pattern and/or an area of the at least one illumination feature may depend on the circle of confusion in the image. The illumination source may comprise the at least one light source configured for generating the at least one illumination pattern. Specifically, for generating and projecting the illumination pattern, the illumination source may comprise at least one laser source and at least one diffractive optical element (DOE). The illumination source may comprise at least one point projector, such as the at least one laser source and the DOE, adapted to project at least one point pattern. As further used herein, the term “projecting at least one illumination pattern” refers to providing the at least one illumination pattern for illuminating the at least one object. The projected illumination pattern may be scarce, as only a single illumination feature, such as a single point, may be present. For increasing reliability, the illumination pattern may comprise several illumination features such as several points.

For example, the illumination source may comprise at least one line laser. The line laser may be adapted to send a laser line to the object, for example a horizontal or vertical laser line. The illumination source may comprise a plurality of line lasers. For example, the illumination source may comprise at least two line lasers which may be arranged such that the illumination pattern comprises at least two parallel or crossing lines. The illumination source may comprise the at least one light projector adapted to generate a cloud of points such that the illumination pattern may comprise a plurality of point pattern. The illumination source may comprise at least one mask adapted to generate the illumination pattern from at least one light beam generated by the illumination source. The illumination source may be one of attached to or integrated into a mobile device such as a smartphone.

The image of the body part is typically received from a camera. The image may be received directly from the camera or indirectly, for example a camera records the image and temporarily stores it on a storage medium, for example in a computer or a cloud from where the image is then received. The camera usually contains a pixelated sensor, such as a CCD or CMOS. The camera is sensitive in at least part of the wavelength range of the illumination source, for example in the near-infrared range. Typically, light from the illumination source is directed towards the body part, the body part reflects the light which is than recorded by the camera. The camera can be part of the device executing the training method of the invention, for example a smartphone, or it is mounted on a different system. Hence, it is possible to receive the image of the body part from a different system, for example a separate camera. It is also possible to receive the image of the body part from a storage system, for example a cloud system, to which the images have been uploaded. This may enable the usage of images in different devices and reuse them for different use cases.

The training method further contains determining skin pattern features from the image. A “skin pattern feature” refers to a pattern feature which has been reflected by skin. Skin pattern features can be determined by making use of the fact that skin has a characteristic way of reflecting light: It is both reflected by the surface of the skin and also partially penetrates the skin into the different skin layers and is scattered back therefrom overlying the reflection from the surface. This leads to a characteristic broadening or blurring of the pattern features reflected by skin which is different from most other materials. This characteristic broadening can be detected in various ways. For example, it is possible to apply image filters to the pattern features, for example a luminance filter; a spot shape filter; a squared norm gradient; a standard deviation; a smoothness filter such as a Gaussian filter or median filter; a grey-level-occurrence-based contrast filter; a grey-level-occurrence-based energy filter; a grey-level-occurrence-based homogeneity filter; a grey-level-occurrence-based dissimilarity filter; a Law's energy filter; a threshold area filter. In order to achieve best results, at least two of these filters are used. Further details are described in WO 2020/187719.

The result when applying the filter can be compared to references. The comparison may yield a similarity score, wherein a high similarity score indicates a high degree of similarity to the references and a low similarity score indicates a low degree of similarity to the references. If such similarity score exceeds a certain threshold, the pattern feature may be qualified as skin pattern feature. The threshold can be selected depending on the required certainty that only skin pattern features shall be taken into account, so minimizing the false positive rate. This comes at the cost of identifying too few pattern features are recognized as skin pattern features, i.e. yield a high false negative rate. The threshold is hence usually a compromise between minimizing the false positives rate and keeping the false negative rate at a moderate level. The threshold may be selected to obtain an equal or close to equal false negative rate and false negative rate.

It is possible to analyze each pattern feature separately. This can be achieved by cropping the image showing the body part while it is illuminated with patterned light into several partial images, wherein each partial image contains a pattern feature. It possible that a partial image contains one pattern feature or more than one pattern features. If a partial image contains more than one pattern feature, the determination if a particular pattern feature is a skin pattern feature is based on more than one partial images. This can have the advantage to make use of the correlation between neighboring pattern features.

The determination of skin pattern features can be achieved by using a machine learning algorithm. The machine learning algorithm is usually based on a data-driven model which is parametrized to receive images containing a pattern feature and to output the likelihood if the pattern feature is skin or not. The machine learning algorithm needs to be trained with historic data comprising pattern features and an indicator indicating if the pattern feature has been reflected by skin or not. Particularly useful machine learning algorithms are neural networks, in particular convolutional neural networks (CNN). The kernels of the CNN can contain filters as described above capable of extracting the skin information out the broadening or blurring of the pattern feature.

The determination of skin pattern features may yield a list of skin pattern features containing the pattern features which have been identified as being reflected by skin. The list may contain the position of the skin pattern features in the image received. Alternatively or additionally, the list may also contain partial images each containing a skin pattern feature.

The training method may further comprise determining a distance for the skin pattern features, wherein the distance indicates how far the area of the body part reflecting the light pattern feature is away from the camera. Such distance information can be used to generate a 3D image of the body part. The distance information may contributed to the accuracy of the data-driven model. The distance information may, for example, contain information about deformations such as swelling of a body part. The distance can be determined from a pattern feature by determining the brightness of the central part of the pattern feature and divide it by the brightness of the peripheral part of the pattern feature and comparing the quotient with a reference. Details of this method are described in WO 2018/091649. Alternatively, the distance can be determined by triangulation or by the stereo method if two images taken from different angles are available. The result is a list or vector containing the distance for the skin pattern features which can be further processed together with the skin pattern features list or separately.

The training method further comprises receiving from a user interface a user-selected parameter value. A “user interface” is any interface allowing a user to input information. The user interface can be a graphical user interface displayed on a display. This display may be part of the device further comprising the illumination source and the camera. The display may also be part of a different system, for example a website hosted in a cloud which is capable of providing the user input data. A user interface may also be a data transfer interface which can receive a file provided by a user, such as an interface to a data storage medium or a communication interface such as a network connection interface.

The “user-selected parameter value” is any information related to the condition of the human. This means that the user-selected parameter value is related to the appearance of the body part as recorded by a camera. Hence, a difference in the user-selected parameter value correlates with a detectable difference of the body part. The user-selected parameter value can be a numerical value, for example a Boolean value, an integer value, or a float value. The user-selected parameter value can also be a vector or a matrix. The user-selected parameter value received from the user interface may originate from measurements or observations. Depending on the use case, a specialized measurement device may be available. For example, if the user-selected parameter value refers to a blood pressure value, the blood pressure value may be obtained from a blood pressure measurement device. It is also possible that the user-selected parameter value is subjectively described, for example a scale value indicating the mood or the perceived wakefulness.

A user-selected parameter value is related to the user-selected parameter. The “user-selected parameter” refers to the meaning of the user-selected parameter value, so what the user-selected parameter value relates to. Examples for user-selected parameters include medical conditions like blood pressure, blood oxygen saturation, menstrual cycle, allergic reactions, status of a chronic disease, stress level, infections; cosmetic conditions like skin moisture, wrinkling, skin fatigue; fitness conditions like level of endurance, oxygen capacity from altitude training.

The user-selected parameter may be preset in the training method of the invention. In this case the user only inputs the user-selected parameter value. Preferably, however, the user can also input the user-selected parameter. In this case the training method of the invention comprises receiving from a user interface a user-selected parameter and the user-selected parameter value related to the condition of the human. The user-selected parameter may be selected from a preset list of user-selected parameters. It is also possible that the user inserts a new user-selected parameter. This allows the user to set up his own use cases independent of what the producer of the device or the provider of the software implementing the training method of the invention has preset. The new user-selected parameter may be appended to a list of existing user-selected parameters. The user-selected parameter may be shared between different users, for example by exchanging the user-selected parameters via a network communication interface. Any entry of a user-selected parameter value may be accompanied by selecting the respective user-selected parameter from the list of user-selected parameters.

It may be useful to use the training method of the invention for more than one user-selected parameter in parallel or consecutively. In this case the training method of the invention comprises receiving from a user interface a user-selected parameter value and a user-selected parameter related to the condition of the human. In this way, each user-selected parameter value can be associated with a specific user-selected parameter. All user-selected parameter values associated to one specific user-selected parameter can hence be treated separately from user-selected parameter values associated with other user-selected parameters.

The list of skin pattern features obtained from the image of the body part is labelled with the corresponding user-selected parameter value. Thereby, a data set is obtained comprising the skin pattern features and the user-selected parameter value. Such data set can be used to train a data-driven model.

“Data-driven model” refers to a mathematical model that is parametrized according to a training data set to reflect the correlations between the skin pattern features and the user-selected parameters value. Data-driven models are set up without reflecting any underlying physical laws of nature. These are taken into account solely by using the correlations in the data. The data-driven model is preferably a data-driven machine learning model. The data-driven model can be a linear or polynomial regression, a decision tree, a random forest model, a Bayesian network, support-vector machine or, preferably an artificial neural network, in particular a convolutional neural network. The same data-driven model may be used for all user-selected parameters. It is also possible to use different data-driven models for certain classes of user-selected parameters. If the user selects a new user-selected parameters, i.e. one which is not preset for example by the provider of the software implementing the training method of the invention, it is not apparent which data-driven model should be used. The training method may therefore comprise receiving from a user interface the data-driven model for training. This can be achieved by a selection of data-driven models prompted to the user to chose from, so the selection is received from the user interface. Alternatively, more than one data-driven models are received, for example from a storage medium. More than one data-driven models, for example at least two or at least three, may then be trained. The trained data-driven models are compared, for example by using some data sets, which have been excluded from the training data sets, as validation data sets. The data-driven model with the lowest deviations from the validation data sets may then be used. This procedure has the advantage that the user does not have to have the technical skills to perform this task by himself.

Training the data-driven model typically involves a plurality of data sets. Depending on the complexity of determining the user-selected parameter value from the skin pattern features, the number of data sets are determined. It is also possible to train the data-driven model with a minimum of data sets, for example 10 or 20, and determine the accuracy of the trained data-driven model. This can be achieved by using data sets which have not been used for training as validation data sets. This means that the model receives the skin pattern features and determines the user-selected parameter value. The result is compared with the user-selected parameter value in the validation data set. If the average difference exceeds a predefined threshold, the user may be invited to provide further data sets. If further data sets do not improve the average difference between determined user-selected parameter value and actual user-selected parameter value, the user may be informed that the specific user-selected parameter can potentially not be determined with the data-driven model.

The training method may further comprise retraining the data-driven model, wherein retraining refers to using new training data sets for an already trained data-driven model. The retaining may include adding the new training data sets to the old training data sets and start training the data-driven model completely. This process may be useful if the data-driven model has insufficient accuracy due to a too low number of training data sets. Alternatively, retraining may include training the data-driven model by using only the new data sets. Such retraining is useful if conditions occur having a permanent influence on the user-selected parameter. An example could be that the user has an accident and has to change his way of life, for example by using a wheelchair. Retraining only using new data sets may lead to a loss of information from the old data sets, also called “forgetting”. This effect may be desirably because the old data sets are not useful any more due to drastic changes. Often, however, more subtle changes occur, so the information of the old data sets should not be completely lost. Therefore, retraining only using new data sets may involve allowing only small changes to the data-driven model which has been trained with the old data sets. This can be achieved, for example, by using a loss function for training which adds a value for a change to the loss function depending on the degree of change. This means that a high change to the data-driven model causes a high additional value to the loss function while small changes only add small values to the cost function. Hence, retraining the data-driven model can adapted to changes influencing the user-selected parameter.

In a preferred aspect, the invention relates to method for training a data-driven model for determining a user-selected parameter value related to the condition of a human comprising:
(a) receiving an image of a body part of the human while the body part is illuminated by a light pattern containing at least one pattern feature,
(b) determining skin pattern features from the image, wherein a skin pattern feature is a pattern feature which has been reflected by skin,
(c1) receiving from a user interface a user-selected parameter value and a user-selected parameter related to the condition of the human,
(c2) generating a data set comprising the skin pattern features with the user-selected parameter value,
(c3) collecting a plurality of the data sets, wherein the plurality of the data sets relates to the same user-selected parameter,
(c4) subdividing the plurality of data sets into a training data set and a validation data set, wherein the training data set contains a part of the plurality of data sets and the validation data set contains a different part of the plurality of data sets,
(d) training a data-driven model with the training data set,
(e) validating the data-driven model with the validation data set.

In another aspect, the invention relates to a method for determining a user-selected parameter value. The method contains receiving an image of a body part of the human while the body part is illuminated by a light pattern containing at least one pattern feature and determining skin pattern features from the image, wherein a skin pattern feature is a pattern feature which has been reflected by skin. These steps correspond to the steps for the training method. Hence, the description including preferred embodiments of the training method apply also for the determination method.

The determination method further comprises determining the user-selected parameter value related to the condition of a human from the skin pattern features by using a data-driven model which has been trained by a set of historic data comprising skin pattern features and the user-selected parameter value. The training of the data-driven model may be executed by using the training method of the present invention. The description including preferred embodiments for the training method hence applies also for the determination method.

The determination method further comprises outputting the user-selected parameter value. Outputting can mean displaying on a display, for example in a graphical user interface. Outputting can also mean writing to a non-transitory storage medium, for example a hard drive or a flash storage medium. Outputting can also mean sending the user-selected parameter to a remote system, for example to a laptop via a WLAN or Bluetooth connection, or a cloud service via a network connection.

Usually, for each user-selected parameter, specialized sensors or measurement devices exist which can determine the user-selected parameter value. However, the advantage of the determination method of the present invention is that different user-selected parameters can be determined with the same hardware. Hence, it is possible to rent such a specialized device for a while to collect training data sets. Once the data-driven model is trained, no specialized measurement devices are necessary anymore.

In another aspect, the invention relates to non-transitory computer-readable data medium storing a computer program including instructions for executing steps of the training method and/or the determination method of the present invention. “Computer-readable data medium” refers to any suitable data storage device or computer readable memory on which is stored one or more sets of instructions (for example software) embodying any one or more of the methodologies or functions described herein. The instructions may also reside, completely or at least partially, within the main memory and/or within the processor during execution thereof by the computer, main memory, and processing device, which may constitute computer-readable storage media. The instructions may further be transmitted or received over a network via a network interface device. Computer-readable data medium include hard drives, for example on a server, USB storage device, CD, DVD or Blue-ray discs. The computer program may contain all functionalities and data required for execution of the method according to the present invention or it may provide interfaces to have parts of the method processed on remote systems, for example on a cloud system. The term “non-transitory” has the meaning that the purpose of the data storage medium is to store the computer program permanently, in particular without requiring permanent power supply.

In another aspect, the invention relates to device for determining a user-selected parameter value. The device can be a stationary device such as a desktop computer or a terminal. The device can also be a portable device such as a smartphone, a tablet, a smartwatch, a laptop or a computing device integrated into an apparatus for physical exercises.

The device contains a projector or illumination source for projecting patterned light containing at least one pattern feature onto a body part of the human, a camera for recording an image of the body part while it is illuminated by patterned light and a user interface. In particular, the device is usable for executing the training method of the present invention. The description and preferred embodiments above for the illumination source, the camera and the user interface apply also for the device.

The device further contains a processor for determining skin pattern features from the image, wherein a skin pattern feature is a pattern feature which has been reflected by skin and training a data-driven model with a training dataset comprising the skin pattern features and the user-selected parameter value. Preferably, the processor is in addition configured for determining the user-selected parameter value related to the condition of a human from the skin pattern features by using a data-driven model which has been trained by a set of historic data comprising skin pattern features and the user-selected parameter value.

The processor may be a local processor comprising a central processing unit (CPU) and/or a graphics processing units (GPU) and/or an application specific integrated circuit (ASIC) and/or a tensor processing unit (TPU) and/or a field-programmable gate array (FPGA). The processor may also be an interface to a remote computer system such as a cloud service. The processor may include or may be a secure enclave processor (SEP). An SEP may be a secure circuit configured to authenticate an active user, e.g. the user that is currently using device. A “secure circuit” may be a circuit that protects an isolated, internal resource from being directly accessed by an external circuit. The internal resource may be memory that stores sensitive data such as personal information, e.g. biometric information or medical information, encryptions keys or random number generator seeds. The internal resource may also be circuitry that performs services/operations associated with sensitive data.

In an embodiment, the condition of the human may be a condition of a human's skin.

In an embodiment, the light pattern containing at least one pattern feature may be a hexagonal or trinclinic dot light pattern.

### BRIEF DESCRIPTION OF THE FIGURES

FIG. 1 shows a device according to one aspect of the invention.

FIG. 2 shows a training method according to another aspect of the invention.

FIG. 3 shows a determination method according to another aspect of the invention.

### DESCRIPTION OF EMBODIMENTS

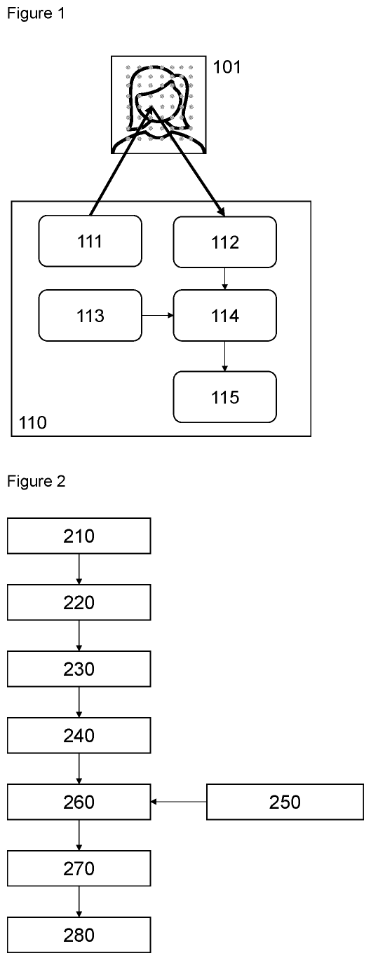

FIG. 1 shows a device for determining a user-selected parameter. The device 110 can be a portable computer device like a smartphone, a tablet, a smartwatch or a laptop. The device 110 contains a projector 111 for projecting a light pattern containing at least one pattern feature. The light pattern may be a cloud of periodic or non-periodic dots, hence a pattern feature may be a dot. The projector may emit in the infrared wavelength range, such as the near-infrared range. The projector may contain a laser and a diffractive optical element to generate the light pattern. The projector may also contain an array of vertical cavity surface emitting lasers (VCSEL) to generate the light pattern. Usually, the projector also contains lenses to focus the light beams onto the body part. The device further contains a camera 112 for recording an image of the body part while it is illuminated by patterned light. The camera 112 is sensitive in at least parts of the wavelength area emitted by projector 111. The device 110 further contains a user interface 113 for receiving a user-selected parameter related to the condition of the human. The user interface may be a display on the device prompting the user to input the user-selected parameter and thus receiving the user-selected parameter.

Both the image from the camera and the user-selected parameter are forwarded to a processor 114 for processing. The processor is adapted for training a data-driven model with labelled data as well as executing the model to retrieve a user-selected parameter. Hence, the processor is configured for determining skin pattern features from the image. The processor is further configured for training a data-driven model with a training dataset comprising the skin pattern features and the user-selected parameter. The processor is further configured for determining the user-selected parameter by using the data-driven model which has been trained by a set of historic data comprising skin pattern features and the user-selected parameter. The device 110 further contains an output 115 to output the user-selected parameter. The output 115 may be a display, for example the same display used as user interface 113 for receiving a user-selected parameter.

FIG. 2 shows a method for training a data-driven model for determining a user-selected parameter. In a first step 210 a body part, for example a face, is illuminated with patterned light containing a least one pattern feature. The patterned light reflected by the body part is recorded by a camera in step 220, hence generating an image of the body part while it is illuminated with patterned light. In step 230, the pattern features are then analyzed to determine whether they are reflected by skin or not. If a pattern feature has been detected as being reflected by skin, it qualifies as skin pattern feature. This analysis is usually performed for each pattern feature separately. The determination yields a list of all skin pattern features 240. A user may input a user-selected parameter (250) which is related to the body part just recorded. For the input, the user may firstly determine, which type of user-selected parameter he wants to enter, for example the blood pressure. Then, the user enters the user-selected parameter, i.e. the value for the type of user selected parameter. For the example for blood pressure, this may be the systolic value in mmHg. The list of skin pattern features 240 is then labelled in step 260 with the user-selected parameter to generate one data set. In step 270 multiple of such data sets are collected by repeating steps 210 to 260. When a certain number of data sets are collected, a data-driven model is trained with these data sets. For this purpose the data-driven model is parametrized according to the data sets, i.e. receives the skin pattern features 240 as input and the user-selected parameter as output. During the training process, the data-driven model is adjusted to best fit the training data sets. Often, it is useful to use some of the data sets as validation data sets in order to verify if the data-driven model yields sufficiently reliable values for the determination of the user-selected parameter. If this is not the case, the user may be invited to add further training data sets by repeating steps 210 to 260. Once, the model is trained, it can be deployed.

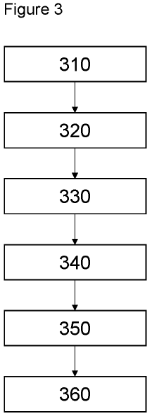

FIG. 3 shows a method for determining a user-selected parameter. In a first step 310 a body part, for example a face, is illuminated with patterned light containing a least one pattern feature. The patterned light reflected by the body part is recorded by a camera in step 320, hence generating an image of the body part while it is illuminated with patterned light. In step 330, the pattern features are then analyzed to determine whether they are reflected by skin or not. The result is a list of skin pattern features 340 as described for the training method above. In step 350 the user-selected parameter is determined by using a data-driven model which has been trained by a set of historic data comprising skin pattern features and the user-selected parameter. The data-driven model receives the list of skin pattern features as input and outputs the user-selected parameter. The thus obtained user-selected parameter can be output in step 360, for example by displaying it on a user interface, for example a display.

### EXAMPLE

The following example is intended to illustrate a potential implementation of the present invention. It shall by no means limiting to the scope of the invention.

A smartphone is equipped with an infrared VCSEL array which can project an illumination pattern of dots in a triclinic arrangement. The pattern is focused by lenses such that a body part in a typical distance to a smartphone of 20 to 60 cm can be illuminated. The pattern illuminates a face with about 50 spots. The smartphone is further equipped with a CMOS camera sensitive in the infrared range. An app or computer program is installed on the smartphone. In this app the user can select a predefined user-selected parameter or create a new one. For this example, the user creates a new user-selected parameter, namely the prediction of a headache attack of a migraine patient. It is known that migraine has a considerable influence on the skin due to differences in blood circulation and accessibility of nutrients for the skin. The user decides to scan his face. The app invites the user to scan his face every day and enter a headache score between 0 (no pain) and 10 (extreme pain) for the same day.

The user scans his face every day for eight weeks. For each scan, the projector is triggered to illuminate the face with the dot pattern. The camera records an image and passes it to the processor. The processor determines the dots corresponding to reflexes on skin on the face. A data set is created with the list of dots reflected by skin and a vector containing the pain value for the day the image is recorded as well as the pain value for the following three days, once these values are entered. When such data sets are collected for the full period of eight weeks, a convolutional neural network is trained by providing the datasets. Some data sets are not used for training, but for validation to make sure that the training yields a useful result. Once the validation is successful, the user gets a message on the display that the model has been trained successfully and can now be used.

For using the model, the user selects in the app the option to use the prediction of a headache attack. The face is scanned as for the training. The skin pattern features are determined, followed by providing the skin pattern features to the trained neural network. The neural network determines as an output a vector of four numbers between 0 and 10 indicating the likelihood and strength of a headache to be expected at the same day and the following three days.

Other variations to the disclosed embodiments can be understood and effected by those skilled in the art in practicing the claimed invention, from a study of the drawings, the disclosure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality. A single unit or device may fulfill the functions of several items recited in the claims. The mere fact that certain measures are recited in mutually different dependent claims does not indicate that a combination of these measures cannot be used to advantage.

1. A method for training a data-driven model for determining a user-selected parameter value related to the condition of a human comprising:
(a) receiving an image of a body part of the human while the body part is illuminated by a light pattern containing at least one pattern feature,
(b) determining skin pattern features from the image, wherein a skin pattern feature is a pattern feature which has been reflected by skin,
(c) receiving from a user interface a user-selected parameter value related to the condition of the human, and
(d) training a data-driven model with a training dataset comprising the skin pattern features and the user-selected parameter value.

2. The method of claim 1, wherein the body part is illuminated by a light pattern in the near infrared region.

3. The method of claim 1, wherein the body part is a face.

4. The method of claim 1, wherein determining skin pattern features comprises cropping the image into several partial images, wherein each partial image contains a pattern feature.

5. The method of claim 1, wherein a convolutional neural network is used for determining skin pattern features.

6. The method of claim 1, wherein the method comprises receiving from a user interface a user-selected parameter related to the condition of the human.

7. The method of claim 1, wherein more than one data-driven models are trained, and the best fitting model is selected.

8. The method of claim 1, wherein the method further comprises retraining the trained data-driven model with new data sets.

9. A method for determining a user-selected parameter value related to the condition of a human comprising:
(a) receiving an image of a body part of the human while the body part is illuminated by a light pattern containing at least one pattern feature,
(b) determining skin pattern features from the image, wherein a skin pattern feature is a pattern feature which has been reflected by skin,
(c) determining the user-selected parameter value related to the condition of a human from the skin pattern features by using a data-driven model which has been trained by a set of historic data comprising skin pattern features and the user-selected parameter value, and
(d) outputting the user-selected parameter value.

10. The methods of claim 1, wherein the condition of the human is a condition of a human's skin.

11. A non-transitory computer-readable data medium storing a computer program including instructions for executing steps of the method according to claim 1.

12. A device for determining a user-selected parameter value related to the condition of a human comprising:
(a) a projector for projecting patterned light containing at least one pattern feature onto a body part of the human,
(b) a camera for recording an image of the body part while it is illuminated by patterned light,
(c) a user interface for receiving a user-selected parameter value related to the condition of the human, and
(d) a processor for
determining skin pattern features from the image, wherein a skin pattern feature is a pattern feature which has been reflected by skin, and
training a data-driven model with a training dataset comprising the skin pattern features and the user-selected parameter value.

13. The device of claim 12, wherein the device is a portable device.

14. The device of claim 12, wherein the processor is further configured for determining the user-selected parameter value related to the condition of a human from the skin pattern features by using a data-driven model which has been trained by a set of historic data comprising skin pattern features and the user-selected parameter value.

15. The device of claim 11, wherein the processor is or comprises a secure enclave processor.

16. The device of claim 12, wherein the device is a smartphone.
